(19) United States
(12) Patent Application Publication
(10) Pub. No.: US 2006/0062835 A1
(43) Pub. Date: Mar. 23, 2006

(54) Multi-disciplinary nicotine craving reduction kit with potentiating homeopathic elements

(75) Inventor: Timothy Weil, Chicago, IL (US)

Correspondence Address: DORT PARTNERS IP PLLC;PATENTS, 1700 Diagonal Road, Suite 300, Alexandria, VA, 22314, US

(73) Assignee: Smoke Free in 3, inc., Chicago (US)

(21) Appl. No.: 11/184,530

(22) Filed: Jul. 19, 2005

Related U.S. Application Data
(60) Provisional application No. 60/589,098, filed on Jul. 19, 2004.

(51) Int. Cl. A61L 15/16; A61H 5/00; A61K 36/899; A61K 36/28

(52) U.S. Cl. 424/448; 601/1; 424/750; 424/757; 424/737

(57) ABSTRACT

A process for assisting in the reduction in the craving of an addictive substance, particularly nicotine is described that includes the following steps: placing at least one pressure pad on a target pressure point on the ear of a patient for a first time period; during a second time period, the patient periodically ingests a craving reduction composition orally; during a third time period overlapping with the first time period, the patient periodically ingests a stress reduction formula in oral form, removing the at least one pressure pad at a time after the first time period and replacing the pressure pad with a replacement pressure pad; lastly, the patient continues with the pressure pad replacement, composition ingestion and oral ingestion of the stress reduction formula until a treatment end time.

### CROSS-REFERENCE TO RELATED APPLICATIONS

This application claims priority to U.S. Provisional Patent Application No. 60/589,098 filed on Jul. 19, 2004, entitled MULTI-DISCIPLINARY NICOTINE CRAVING REDUCTION KIT USING POTENTIATING ELEMENTS AND METHOD FOR USE by Timothy Weil, which is incorporate by reference herein, for all purposes.

### STATEMENT REGARDING FEDERALLY SPONSORED RESEARCH OR DEVELOPMENT

Not applicable.

### BACKGROUND OF THE INVENTION

Smoking or nicotine cessation has many challenges, including physical, emotional and biochemical challenges. While there are many smoking and nicotine cessation methods in the marketplace, they generally have not been effective in addressing all of the challenges experienced by persons who desire to quit smoking. Consequently, those desiring to quit smoking must use multiple cessation systems, and often generally drugs to aid in their quest to stop smoking.

Therefore, a need exists for a smoking cessation program that is comprehensive and directly addresses each of the three major categories of symptoms or challenges experienced during smoking cessation.

### SUMMARY OF THE INVENTION

Briefly stated, a process for assisting in the reduction in the craving of an addictive substance, specifically nicotine, is provided that comprises at treatment time, placing at least one pressure pad on at least one target pressure point on the ear of a patient for a first time period, first time period being the treatment duration, preferably two target pressure points of a set of five or six pressure points that are generally used for craving reduction; during a second time period overlapping with the first time period, the patient periodically ingests a craving reduction composition orally (the composition is in spray form and includes at least one set of nutrients); during a third time period overlapping with the first time period, the patient periodically ingests a stress reduction formula in oral form. Next the patient removes at least one pressure pad at a time after the first time period and replaces the pressure pad with a replacement pressure pad. Lastly, the patient continues the pressure pad replacement, composition ingestion and oral ingestion of the stress reduction formula until a treatment end time.

In another aspect of the invention, a nicotine reduction kit is provided that consists of the following items e: a homeopathic remedy for reducing stress and anxiety associated with smoking cessation; an herbal nutrient in oral spray form for providing nicotine detoxification nutrients; and a set of acupressure ear pads, each of the pads with a metal bead in the center. Each of the above features is configured for simultaneous use.

In yet another aspect of the invention, a nicotine reduction kit is provided that consists these following elements: a homeopathic remedy (HR) in oral pellet form for reducing stress and anxiety associated with smoking (nicotine) cessation; an herbal nutrient in oral spray HS form for providing nicotine detoxification nutrients to a smoking cessation patient; a set of acupressure ear pads AP, each of the pads with a metal bead in the center; and lastly, an optionally supplemental nutrient for ingestion, wherein the supplemental nutrient includes either green tea or an amino acid, or both.

In a further aspect of the invention, a method for a reduction in the dependency of addictive substances for a patient is provided that comprises the following: placing a plurality of ear squares onto the patient's ears (generally applied to one ear), wherein at least one ear square is applied to each of the patient's ears, wherein the ear squares are placed on the patient's ear, preferably messaged onto the patients' ear; spraying an herbal spray into the patient's mouth, wherein the spraying of the herbal spray is repeat 2-10 times per day; and lastly, ingesting at least one homeopathic pellet.

### BRIEF DESCRIPTION OF THE DRAWINGS

The invention can be better understood by reference to the following illustrative and representative drawings, which describe the elements of relationships between the particular embodiments of the invention.

FIG. 1 shows a triangular representation of cessation needs.

FIG. 2A illustrates an alternate representation of a patient's needs with “crossover” or multi-disciplinary need or challenge zones.

FIG. 2B shows an alternate patient's needs or challenges with a greater emphasis on one of the challenge areas.

FIG. 3 represents an embodiment of the invention in which a patient's cessation needs are addressed by particular elements of a preferred embodiment of the invention.

FIG. 4 illustrates an alternate embodiment of invention in which the Naturopathic Triangular Technique™ or NTT™ is supplemented by an additional feature.

FIG. 5 shows a customized NTT™ system.

### DETAILED DESCRIPTION OF THE INVENTION

The preferred embodiment of the present invention takes the form of a multi-disciplinary kit under the trade name SMOKE FREE IN 3™. The invention is a stop smoking cessation aid and supplemental support system that utilizes at least a three-fold synergistic interdisciplinary system known by the trade name Naturopathic Triangular Technique (NTT™). The NTT™ assists patients in their efforts to quit using addictive substances, nicotine in particular.

The NTT™ combines at least three synergistic techniques or elements to fight the challenges associated with the cessation of addictive substance use, particularly nicotine, and the quitting smoking process, in particular. The combined consequence of these synergistic techniques or elements together is more effective than any one technique and more effective than techniques in an unconfigured combination.

The defining characteristic that distinguishes particular embodiments of the invention over the prior art is that the NTT™ system directly addresses the issues smokers (or other substance abusers) encounter while quitting. Each component of the program directly addresses each of the three major categories of symptoms experienced during smoking cessation: (1) physical, which generally corresponds to physical withdrawal symptoms encountered by the smoker, (2) emotional, which generally corresponds to the stress and anxiety produced by smoking cessation, and (3) biochemical, which generally corresponds to the cellular chemistry or nutritional needs. Thus, the NTT™ can be generally considered comprehensive and synergistic cessation system, which does not need to be used in conjunction with other smoking cessation aids, such as allopathic physician-prescribed medicines like Zyban® or Wellbutrin®. However, the present invention may be used in conjunction with pharmaceutical methods with supervision and approval of the patient's physician.

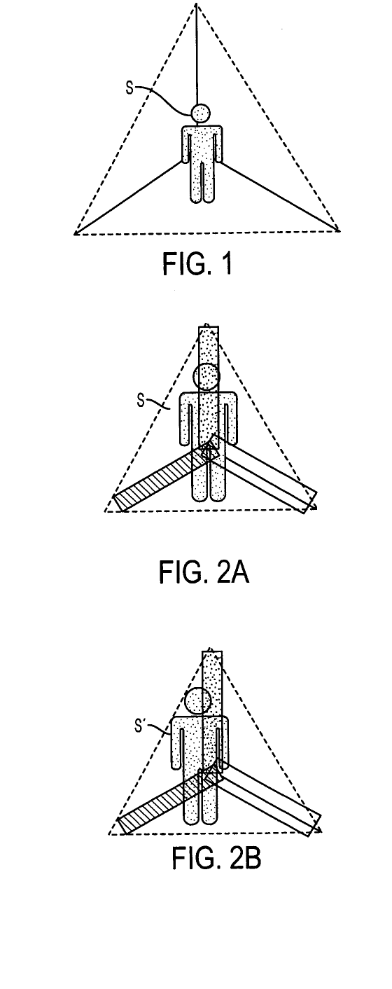

FIG. 1 illustrates a triangular representation of a patient's S needs or challenges during the cessation process, and is particularly applicable and/or useful in considering nicotine or smoking cessation. The triangle is divided into three representational zones that indicate the potential problems involved in smoking cessation and include physical, which generally corresponds to the patient's physical withdrawal symptoms, emotional, which generally corresponds to the patient's stress and anxiety associated with smoking cessation and biochemical, which generally corresponds to the patients nutritional or cellular needs “zones” (all parts of the triangle are labeled as such) that may be addressed to effectively assist the patient P in the process of cessation from dependency to addictive substance.

To effectively assist the patient in the process of cessation from dependency to addictive substances, a novel process has been invented. It consists of the following events: the process comprises placing at least one pressure pad on at least one target pressure point on the ear of the patent for a treatment period at a treatment start time; overlapping with the treatment period, the patient periodically ingests a craving reduction composition orally, the composition in spray form includes at least one set of nutrients; also during a time period that overlaps with the treatment period, the patient periodically ingests a stress reduction formula in oral form; during the treatment period if at least one pressure pad is removed it is replaced with a replacement pressure pad; and the pressure pad replacement, composition ingestion and oral ingestion of the stress reduction formula is continued until a treatment end time.

A novel method for reducing the dependency of addictive substances for a patient has also been discovered. The method comprises placing a plurality of ear squares onto the patient's ears, spraying an herbal spray into the patient's mouth, and ingesting at least one homeopathic pellet. The ear square is applied to each of the patient's ears and generally massaged onto the patient's ears. The spraying of the herbal spray is generally repeated 2-10 times per day, but other application times and frequencies may be employed.

Although it is contemplated that particular embodiments of the invention will be particularly effective in smoking cessation, the psychoactive properties of nicotine also have many properties in common with other addictive licit and illicit substances, particularly stimulants such as, caffeine, cocaine, amphetamines and derivates of amphetamines. Thus, the use of the expression nicotine in a preferred embodiment may also be applicable for recovery from many other addictive substances as well. As such, the inventive kits, programs and methods described herein are all directed to methods for reducing the dependency of addictive substances generally for a patient in specific embodiments of the invention.

FIG. 2A is also useful in understanding particular uses and implementations of the present invention for addictive substance cessation. In FIG. 2A, the physical (in the form of physical withdrawal symptoms or challenges), emotional (in the form of stress and anxiety produced in the patient from smoking cessation), and biochemical (in the form of nutritional or cellular) needs of the smoking cessation patient S, overlap in particular regions P/BC, E/P and E/BC. This illustration can be appreciated by those skilled in the art to understand that particular patient needs during smoking cessation, as discussed in the present disclosure, are meant to be broadly defined and are useful in considering and planning the overall homeopathic treatment of the newly recovered nicotine-dependent patient. Thus, a particular element as illustrated in the present invention may address some physical needs of the patent S, but also overlap to address some emotional needs as well. The overlap of solutions is not meant to reduce the scope of the embodiments of the invention, in which particular elements each primarily addresses one of the three clearly identified areas of the NTT™ triangle, such as biochemical needs, but should also not be considered as limited to addressing only those needs.

FIG. 2B further illustrates the advantages of particular embodiments of the invention in addressing a patient needs and in alternate embodiments of the invention, customization of smoking cessation kits address the varied needs of particular patient. The representative alternate patient S′ in FIG. 2B is located across a different set of “need regions” than the representative patent S in FIG. 2A, who is “equilaterally” divided between the physical, biochemical, and emotional regions, which correspond to the particular challenges of the patient, as discussed above. In contrast, the alternate patient S′ in FIG. 2B, is located “off center” such that the alternate patient's emotional needs, or the part of the alternate patient in the emotional needs region is much less than that of the patient S represented in FIG. 2A. Thus each smoking cessation kit may be individually tailored to the needs of the particular patient, in order to improve the probability of smoking cessation and subsequent maintenance.

In another embodiment, the invention includes one at least two week program that utilizes the Naturopathic Triangular Technique™ (NTT™) to address and combat each of the three major respective challenges that are experienced by the patient during the cessation process. Each of the three main components that make up the Smoke Free in 3 NTT™ kit or system are designed to focus more heavily on one of these three major challenges. It can be appreciated by skilled artisans that there may be some overlap between each of the challenges or cessation needs of the patient and the corresponding solution as implemented in particular embodiments of the present invention. As such, in one embodiment of the invention, the time between the treatment start time and the end of treatment is at least four weeks.

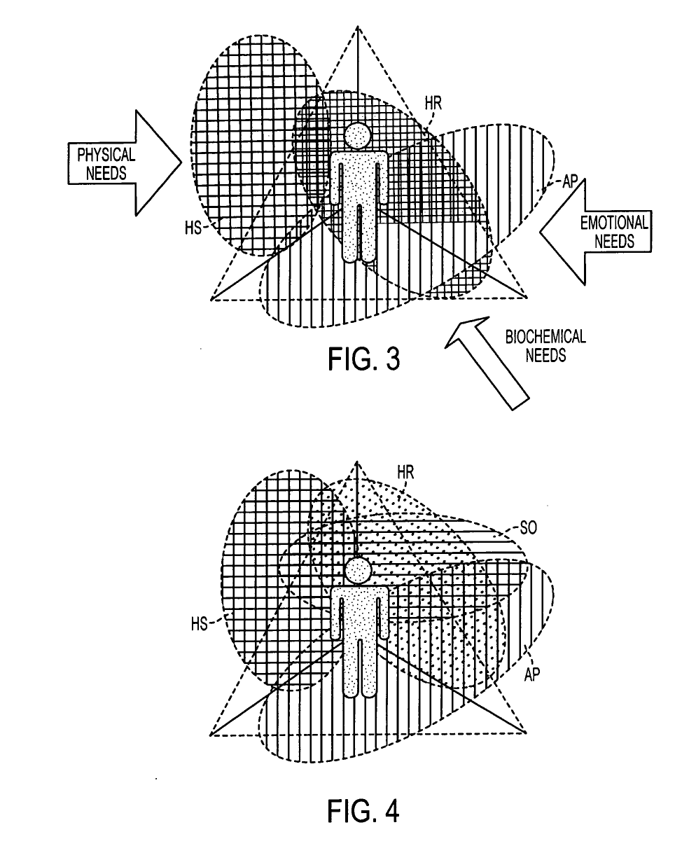

As stated earlier, each component of the methods and systems of the invention directly address each of the three major challenges experienced during smoking cessation. The all-natural or homeopathic Smoke Free in 3™ system is generally easy to follow and takes almost no time to use. The three essential elements include a first element that includes a homeopathic remedy (HR in FIGS. 3-5), which is formulated for smoking cessation. The homeopathic remedy HR reduces the stress and anxiety associated with smoking cessation. The representative second and third components are the (2) oral herbal spray (HS in FIGS. 3-5), and (3) acupressure ear pads (AP in FIGS. 3-5) that have a metal bead on the center which support the patient's body throughout the cessation process and which primarily address the physical withdrawal symptoms or challenges of smoking cessation.

FIG. 3 illustrates the inter-disciplinary and potentiating interactions of the elements of the smoking cessation kit in the form of a representational diagram of the Naturopathic Triangular Technique™. The NTT™ addresses the three-pronged cessation needs of the smoker S (located in the center of the triangle) as shown in FIGS. 1 and 2 above. The first component of the preferred embodiment takes the forms of an herbal spray or HS.

The proprietary herbal spray formula HS is generally in liquid form and comes in a convenient pump spray bottle to facilitate proper use. The herbal spray HS formula promotes detoxification of the body and provides energy and nutrients. The herbal spray primarily address the biochemical BC needs of the patient S, but will also overlap with other needs, such as physical ones (shown in the triangle). The representation of the herbal spray HS ellipse in FIG. 3 is shown overlapping other needs as well for illustrative purposes only, and should not be considered to be a limitation of this particular feature of the preferred embodiment.

The spray HS is formulated to help rebuild the nervous system and reduce nicotine cravings. It also supplies nutrition to increase energy and support cellular function. The spray HS may comprise at least one of the following ingredients: wheat grass, alfalfa, soy, vitamin E, Echinacea and/or beledonia in certain embodiments.

The spray may be used by the patient as needed. In a specific embodiment, the spray is used 2-10 times per day. In another specific embodiment, the spray is used 4-5 times per day.

In one embodiment the spray is formulated and administered as follows:
(0.3332 ml);
Servings per container (in kit): 90;
Proprietary extract blend 29 mg*;
Ingredients: Avena Sativa, Licorice Root, Plantain Leaf & Corm, American Skullcap, Calamus Root, Sassafras Root and Lobelia extract in spring water and 40-45 USP alcohol, glycerin and peppermint essential oil.

The second element of the primary embodiment is the Homeopathic Remedy HR as shown in FIG. 3, which primarily addresses the emotional needs (as shown in the NT™ triangle, of the patient S during the smoking cessation process. The Homeopathic Remedy HR formula (as discussed below) is in pellet form in a preferred embodiment and comes in a small and easy to carry tube to facilitate proper use. Generally, and in one embodiment of the invention, the pellets are ingested by being placed under the tongue. The formula of the homeopathic remedy HR aids in smoking cessation withdrawal symptoms and nicotine cravings and helps to reduce the mental and emotional stress associated with smoking withdrawal. Additionally, the homeopathic remedy HR helps to diminish irritability, anxiety and nervousness. In one specific embodiment of the invention, the pellets HR are ingested by the patient immediately after use of the herbal spray HS of the invention. It should be understood, however, that the steps of the method of the invention may be carried out in any order, in accordance with the invention.

In one embodiment of the invention, active ingredients of the primary homeopathic remedy HR include at least two of the following ingredients:
Alfalfa 12×
Arsenicum album 12×
Caladium seguinum 12×
Daphne indica 12×
Eugenia jambosa 12×,
Kali phosphoricum 12×
Lobelia inflata 12
Nux vomica 12×
Plantago major 12×
Tabacum 12×

The homeopathic remedy HR includes all of the above-listed ingredients in a preferred embodiment.

In another embodiment of the invention, the homeopathic pellet HR comprises at least one of: yeast, anis, phenel, vitamin B, and zinc and grape seed. These ingredients may be in addition to the active ingredients of the homeopathic remedy HR as listed above.

The patient may ingest the number of pellets of the homeopathic remedy HR necessary for that particular patient, and in a specific embodiment of the invention ingests two pellets.

The third element included in the present invention is Pressure Point Ear Squares which are indexed as AP in FIGS. 3-5. In specific embodiments of the invention, the ear squares AP comprise self-adhesive material with a small metal bead adhered thereto. Using the ear squares AP helps give the patient going through the cessation process a feeling of overall wellness and also provides distraction from nicotine cravings. In a specific embodiment of the invention, the ear squares further comprise a fabric patch. The size of the fabric patch is variable and dependent on the size of the accompanying ear square or pad. In a specific embodiment, the fabric patch is 0.5″×0.5″. The ear squares are generally applied approximately to the top portion of the patient's ear. In a specific embodiment of the invention, two ear squares are applied to each of the patient's ears. In particular embodiments of the invention, another ear square is applied to the front portion of the patient's ear. The pressure point ear squares AP are used in traditional Chinese medicine to help reduce cravings for nicotine, reduce stress, balance body chemistry and promote better sleep.

FIG. 4 illustrates an alternative embodiment of the invention, which is a kit that includes licorice root sticks or an alternate oral stimulus SO shown in FIG. 4. The trade names for these embodiments include SUPPLEMENTAL NATUROPATHIC TRIANGULAR TECHNIQUE™ or SUPPLEMENTAL NTT™. In this embodiment, the patient performs oral replacement exercises and continues the exercises until the end of treatment. The sticks are raw licorice root, and are meant to be chewed. The patient may chew on any root of a craving reduction plant family, and does so in a specific embodiment of the invention. When the patient has the urge to reach for a cigarette they can grab a licorice root stick instead and chew on it. The licorice root sticks taste good to most patients and give the patient's hands and mouth something to do, which is often a concern during smoking cessation. Alternates to the licorice root sticks, which act as the oral stimulus or exercise, SO, should provide similar features to facilitate use by the patient S, including providing a pleasant taste, smell and consistency that promotes oral stimulation.

The licorice root sticks are used in place of cigarettes to help break the hand-to-mouth habit of cigarette smoking and thus provide an “alternate oral exercise” or “stimulation.” The sticks can be chewed all day long and will help soothe irritated tissue in the lungs.

Other optional elements of the particular embodiments of the present invention in the SUPPLEMENTAL NTT™ are shown in FIG. 4 and include a Green Tea Supplement GT and/or and Amino Acid Supplement AA, which facilitate the patient's cessation process. The Green Tea Supplement GT supplement is designed to help reduce the increase in appetite associated with quitting smoking and aids in ingestion. Thus, it helps control weight gain. In addition, other ingredients in the formula balance sugar levels to further reduce hunger and appetite. Green Tea by itself is known to reduce mental stress and anxiety.

Representative features of the supplements suitable for use in the present invention are shown below:

Green Tea Supplement
Serving Size: 2 capsules

Servings per Container: 30

| | Amount | % Daily |
|---|---|---|
| | Per Serving | Value |
| Magnesium (Glycinate) | 18 mg | 5% |
| Chromium (Picolinate) | 12 mcg | 10% |
| GARCINIA CAMBOGIA | 500 mg ** | |
| APPLE PECTIN | 250 mg ** | |
| GREEN TEA | 250 mg ** | |
| GYMNEMA SYLVESTRE | 250 mg ** | |
| GINGER ROOT | 100 mg ** | |
| L-GLYCINE | 200 mg ** | |

** Daily Value not established

Other ingredients: gelatin, magnesium stearate, silicone dioxide

Amino Acids & Milk Thistle Supplement
Serving Size: 2 capsules

Servings per Container: 30

| | Amount | % Daily |
|---|---|---|
| | Per Serving | Value |
| Vitamin C | 250 mg | 416% |
| Vitamin E | 100 IU | 333% |
| N-ACETYL-CYSTEINE | 300 mg ** | |
| MILK THISTLE | 250 mg ** | |
| L-TAURINE | 200 mg ** | |
| L-GLUTAMINE | 100 mg ** | |
| L-GLYCINE | 100 mg ** | |
| BILBERRY | 100 mg ** | |

Other ingredients may include gelatin, rice flour, and magnesium stearate and silicone dioxide.

The Amino Acids & Milk Thistle Supplement formula, shown as AA in FIG. 4, was created to help regenerate and rebuild damaged lung cells caused by cigarette smoke. The Amino Acids & Mild Thistle helps the liver process and it detoxifies the body of nicotine and cigarette chemicals. It also has anti-oxidant properties to protect the body from further damage. Representative features of the Amino Acid supplement are shown above.

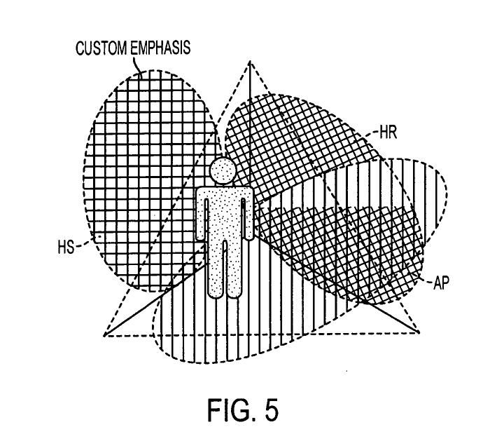

FIG. 5 represents an alternate embodiment of the invention, a customized smoking cessation kit, or CUSTOMIZED NTT™. Customized cessation kits may be more appropriate for the alternate patient S′ as shown in FIG. 2B who may have smoking cessation needs which do not match the needs of a standard patient S in FIG. 2A. FIG. 5A represents an alternate smoking cessation kit for the alternate patient S′ in which the element that addresses the physical needs of the alternate patient S′ is emphasized and the element that addresses the emotional needs of the alternate patient S′, such as the homeopathic remedy HR is de-emphasized. The second alternate patient with great emotional smoking cessation needs unlike the first alternate patient will have a kit with greater emphasis on the emotional component of the NTT™. Thus, the present invention is not limited to a particular set of dosages or amounts, which may be useful to the majority of patients, but can clearly be customized to the cessation needs of each patient.

The present invention emphasizes the synergistic effect of the multiple components to address the comprehensive needs or challenges involved during the withdrawal or cessation period from an addictive substance, particularly nicotine.

The above examples are not meant to be limiting, but illustrative of the features of the present invention. Alternate homeopathic formulas for components of the invention may be substituted without departing from the scope and spirit of the invention, if they provide the particular features of the below defined claims.

1. A process for assisting in the reduction in the craving in a patient of an addictive substance, particularly nicotine, comprising:
at a treatment start time, placing at least one pressure pad on at least one target pressure point on the ear of the patient for a treatment time period;
during a time period overlapping with the treatment time period, the patient periodically ingests a craving reduction composition orally, the composition is in spray form and includes at least one set of nutrients;
during a time period overlapping with the treatment time period, the patient periodically ingests a stress reduction formula in oral form;
replacing the at least one pressure pad at a time during the treatment time period and if the at least one pressure pad is removed from the ear.
continuing the pressure pad replacement, composition ingestion and oral ingestion of the stress reduction formula until a treatment end time.

2. The method as recited in claim 1, further comprising performing oral replacement exercises and continuing oral replacement exercises until the treatment end time.

3. The method as recited in claim 1, wherein the time between the treatment start time and treatment end is at least two weeks.

4. The method as recited in claim 3, wherein the time between the treatment start time and treatment end is at least four weeks.

5. The method as recited in claim 1, wherein the herbal spray is applied 2-10 times per day.

6. A nicotine reduction kit consisting essentially of:
a homeopathic remedy for reducing stress and anxiety associated with smoking cessation;
a herbal nutrient in oral spray form for providing nicotine detoxification nutrients; and
a set of acupressure ear pads, each of the pads with a metal bead in the center;
wherein each of the above features is configured for simultaneous use.

7-8. (canceled)

9. A method for a reduction in the dependency of addictive substances for a patient comprising:
placing a plurality of ear squares onto the patient's ears, wherein at least one ear square is applied to each of the patient's ears, wherein the ear squares are massaged on the patient's ear;
spraying an herbal spray into the patient's mouth, wherein the spraying of the herbal spray is repeat 2-10 times per day; and
ingesting at least one homeopathic pellet.

10. The method of claim 9, wherein the ingesting of the homeopathic pellet is performed immediately after the spraying of the herbal spray.

11. The method of claim 9, wherein the ingesting of the homeopathic pellet comprises ingesting two homeopathic pellets.

12. The method of claim 9, wherein the ingesting of the homeopathic pellet comprises putting the homeopathic pellets under the patient's tongue.

13. The method of claim 9, wherein the homeopathic pellet comprises at least one of the groups consisting of: yeast, anis, phenel, vitamin B, zinc and grape seed.

14. The method of claim 9, wherein the herbal spray comprises at least one of the groups consisting of: wheat grass, alfalfa, soy, vitamin E, echinacia, and/or beledonia.

15. The method of claim 9, wherein the spraying of the herbal spray is repeated 4-5 times per day.

16. The method of claim 9, wherein the ear squares further comprises a fabric patch approximately 0.5″×0.5″.

17. The method of claim 9, wherein the ear squares comprise self-adhesive material and a metal bead.

18. The method of claim 9, wherein two ear squares are applied to each of the patient's ears.

19. The method of claim 18, wherein one ear square is applied approximately in the top portion of the patient's ear and one ear square is applied approximately in the front portion of the patient's ear.

20. The method of claim 9, wherein the addictive substance is nicotine.
